(19) United States
(12) Patent Application Publication
(10) Pub. No.: US 2006/0193795 A1
(43) Pub. Date: Aug. 31, 2006

(54) Appetite suppressant mouth spray

(76) Inventor: Arthur Zuckerman, New York, NY (US)

Correspondence Address: EZRA SUTTON, P.A., Suite 201, 900 Route 9, Woodbridge, NJ, 07095, US

(21) Appl. No.: 11/066,876

(22) Filed: Feb. 25, 2005

(51) Int. Cl. A61K 8/97; A61K 36/254; A61K 36/67; A61K 36/752; A61K 36/328; A61K 36/258; A61K 31/60

(52) U.S. Cl. 424/58; 424/725; 424/728; 424/734; 424/736; 424/748; 424/762; 514/159

(57) ABSTRACT

An appetite suppressant mouth spray formulation which simultaneously suppresses the user's appetite while promoting intraoral health. The mouth spray composition includes mouth spray base ingredients; and a plurality of appetite suppressant and appetite depressant herbs. The mouth spray base ingredients include a combination of Water; Glycerin; Xylitol; Propylene Glycol; Sodium Benzoate, Wintergreen Oil and a flavoring agent. A plurality of appetite suppressing and depressing herbs selected from the group consisting of Griffonia Simplicofolia; Garcina Cambogia; Kola Nut; Guarana; Yerba Mate; Myrrh Gum; Citrus Aurantium; Gymnema Sylvestre and Green Tea.

### FIELD OF THE INVENTION

The present invention relates to a mouth spray and, more particularly, to a formulation of an appetite suppressant oral composition in the form of a mouth spray comprising ingredients which co-act to control the appetite and permit reduction in body weight by spraying the teeth and oral mucosa of a user with the composition.

It has been found that the combination of up to 27% by weight of natural herbs functions as an appetite suppressant agent in a standard mouth spray formulation. This unexpected result from the novel mouth spray composition of the present invention to suppress appetite and promote weight loss provides a new secondary benefit to the promoting intraoral cleanliness with a mouth spray.

### DESCRIPTION OF THE PRIOR ART

Numerous types of appetite suppressants have been provided in the prior art. Current products to suppress appetite and control weight are generally drugs with undesirable side effects, often with a propensity to be addictive; whereas the instant novel appetite suppressant mouth spray provides a non-pharmacological means to suppress the appetite of a user by adding natural herbs to a standard mouth spray.

There are many appetite suppressant products on the market, both prescription items and over the counter products. Most of these products act as central nervous system stimulants, such as amphetamines, or have a similar mechanism of action. Many of the over the counter products, such as Acutrim, Dexatrim, Appedrine, etc. contain Phenylpropanolamine HCl+Caffeine. Ayds, another well known appetite suppressant product, is a caramel candy containing Benzocaine.

Oral compositions which contain natural herbs for temporary relief of pain in the oral cavity are well known products. Such products are readily found on the market and include Anbesol gel, a liquid comprising phenol, alcohol and Benzocaine; Chloroseptic lozenge containing menthol-benzocaine, corn syrup, glycerine, sucrose and flavor for temporary relief of sore throat pain; and Hurricane Topical anesthetic aerosol spray for controlling oral pain.

For example, U.S. Pat. Nos. 3,856,942 and 5,913,894 are illustrative of such prior art. However, none of the above cited prior art disclose an appetite suppressant formulation for controlling weight, comprising a suppressing agent containing natural herbs. These prior art references also do not provide an appetite suppressant in a dental vehicle which may be in the form of a mouth spray. The concept of a mouth spray formulation containing an appetite suppressing agent is novel. While these units may be suitable for the particular purpose to which they address, they would not be as suitable for the purposes of the present invention as heretofore described.

U.S. Pat. No. 3,856,942 discloses an appetite control composition which is readily ingested, comprising a candy base of sucrose and maltose, caffeine, Natural herbs, vitamins and optional flavors including quinine to reduce the sweetness of the sucrose and maltose. This composition is preferably a slow-dissolving hard candy or tablet, but may also be in the form of gum drops, chocolate bars or drops, cotton candy, lozenges or gelatin desserts, all of which are ingestible.

U.S. Pat. No. 4,913,894 discloses an appetite suppressant oral composition containing benzocaine, high impact flavor and a sweetening agent, in the form of a toothpaste. It discloses a method of reducing appetite and is intended for controlling weight of consumers, by applying to the oral cavity a high impact flavor in a toothpaste.

### SUMMARY OF THE PRESENT INVENTION.

The present invention relates to a formulation of an appetite suppressant oral composition in the form of a mouth spray comprising ingredients which co-act to control the appetite and permit reduction in body weight by spraying the teeth and oral mucosa of a user with the composition, along with having an anti-bacterial, anti-plaque forming, and anti-inflammatory effect.

A primary object of the present invention is to provide an appetite suppressant mouth spray that will overcome the shortcomings of prior art products.

The present invention provides an appetite suppressant mouth spray intended to effect weight control. The mouth spray is formulated by adding an appetite suppressing agent comprising up to 27% by weight of natural herbs to a standard mouth spray. Appetite suppression and weight control can be obtained by spraying the teeth and oral mucosa, before each meal, on a regular basis, such as three times per day, with the appetite suppressant mouth spray of the present invention.

Accordingly, another object of present invention is to provide an appetite suppressant mouth spray formulation by the incorporation of an appetite suppressant agent comprising natural herbs.

A further object of the present invention is to provide an appetite suppressant mouth spray which is able to reduce the appetite of a user after spraying the user's mouth with the mouth spray.

A further object of the present invention is to provide an appetite suppressant mouth spray in the form of an oral hygenic liquid for use preferably prior to meals, to promote weight loss.

Another object of the present invention is to provide an anti-bacterial, anti-plaque forming, and anti-inflammatory effect.

Another object of the present invention is to provide an appetite suppressant mouth spray for promoting weight loss as well as good dental hygiene.

Another object of the present invention is to provide an appetite suppressant mouth spray formed from a novel essentially non-ingested composition containing safe natural herbs able to suppress the appetite and promote weight loss of the user.

Another object of the present invention is to provide an appetite suppressant mouth spray that is simple and easy to use.

Another object of the present invention is to provide an appetite suppressant mouth spray that is economical in cost to manufacture.

Additional objects, advantages and novel features of the invention will be set forth in part in the description which follows, and in part will become apparent to those skilled in the art upon examination of the following.

### DETAILED DESCRIPTION OF THE PREFERRED EMBODIMENT

Appetite suppressant mouth spray formulations which simultaneously suppress the users appetite while promoting intraoral cleanliness by acting as an anti-bacterial, anti-plaque forming, and anti-inflammatory effect, are disclosed by the present invention. The mouth spray composition includes mouth spray base ingredients; a plurality of appetite suppressant and appetite depressant herbs. The mouth spray base ingredients include a combination of Water 53% or more; Glycerin 10%; Xylitol 4%; Propylene Glycol 3%; Sodium Benzoate less than 1% and Wintergreen Oil 2% . The appetite suppressing and depressing herbs include Griffonia Simplicofolia 1%; Garcina Cambogia 4%; Kola Nut 4%; Guarana 4%; Yerba Mate 4%; Myrrh Gum 1%; Citrus Aurantium 4%; Gymnema Sylvestre 4% and Green Tea 1%. Myrrh is effective because it acts as an anti-microbial, an anti-fungal, an astringent, and an anti-inflammatory. Green Tea is also effective because of its anti-plaque characteristics.

To achieve the objects in accordance with the present invention, the appetite suppressant mouth spray comprises up to 27% by weight of natural herbs. The amount of natural herbs effective in minimizing the desire for food and suppressing the appetite of a user is up to 27% by weight.

Another component in the appetite suppressant mouth spray composition may be a high, impact flavor which is an intense flavor including, but not limited to, any or all of oil of wintergreen, chocolate, cherry, strawberry, grape and other hunger minimizing flavors. The high impact flavor is a blend of a number of components so that the resultant mixture is a well-rounded, smooth flavor with an intense immediate initial impact, a lasting pleasant aftertaste (e.g. preferably at least about 45-60 seconds) and a prolonged pleasantly smelling impact on the breath of the user. Other good grade artificial flavors may be used provided they are intense flavors of high initial impact with a lasting pleasant aftertaste.

Organic surfact-active agents are preferably used in the composition of the present invention to assist in achieving thorough and complete dispersion of the compositions of the present invention throughout the oral cavity and render the composition more cosmetically acceptable. The organic surface-active agent material may be anionic or nonionic, in nature, and it is preferred to employ as the surface-active agent a detersive material which imparts to the composition detersive and foaming properties. Suitable anionic surfactants include water-soluble salts of higher fatty acid monoglyceride monosulfates, such as the sodium salt of the monosulfated monoglyceride of hydrogenated coconut oil fatty acids, higher alkyl sulfates, such as sodiuim lauryl sulfate, alkyl aryl sulfonates, such as sodium dodecyl benzene sulfonate, higher alkyl sulfoacetates, higher fatty acid esters of 1,2-dihydroxy propane sulfonates, olefin sulfonates and the like.

Other particularly suitable surface active materials include nonionic agents such as condensates of sorbitan monostaerate with approximately 20 moles of ethylene oxide; condensates of ethylene oxide with propylene oxide; condensates of propylene glycol (Pluronics); condensation products of an alpha-olefin oxide containing 10-20 carbon atoms, a polyhydric alcohol containing 2 to 10 carbons; and 2 to 6 hydroxyl groups and either ethylene oxide or a heteric mixture of ethylene oxide and propylene oxide. The resultant detergents are heteric polymers having a molecular weight in the range of 400 to 1600 and containing 40% to 80% by weight of ethylene oxide, with an alpha-olefin oxide to polyhydric alcohol mole ratio in the range of 1:1 to 1:3. The amount of anionic or nonionic surfactant constitutes about 1-3% by weight of the mouth spray formulation.

The mouth spray of this invention may also contain conventional additional ingredients such as coloring or whitening agents and preservatives. These additional ingredients may each be added to the mouth spray in minimal amounts of up to 5% by weight, and preferably up to 1%, provided they do not interfere with the appetite suppressant and compatibility properties of the finished product.

The mouth spray of this invention is prepared by conventional methods of making oral hygenic liquids. More specifically, the gelling agent such as a cellulose gum is dispersed in glycerine, to which is added an aqueous solution containing the sweetening agent such as xylitol, followed by the addition of sorbitol and mixing for a period of about 20 minutes to hydrate the gum, mixing the gum mixture with the polishing agent in a mixer under a vacuum of 28-30 inches of pressure. Lastly, the flavor, the surfactant and natural herbs are added to the vacuum mixer, mixed for a period of about 15 minutes, and the final mixture is placed in a tube. In the practice of this invention to suppress appetite, control weight and simultaneously promote oral hygiene, the mouth spray according to this invention is applied regularly to the oral cavity by spraying the teeth or spraying the mouth for up to 30 seconds, at least three times a day, preferably prior to eating meals or before eating snacks.

The following embodiment is illustrative of the nature of the present invention, but it is understood that the invention is not limited thereto. The composition is prepared in the usual manner and all amounts and proportions referred to herein and in the appended claims are by weight unless otherwise indicated. The preferred embodiment of the appetite suppressant mouth spray is:

PREFERRED EMBODIMENT OF
APPETITE SUPRESSANT MOUTH SPRAY

| INGREDIENTS | PERCENT % |
|---|---|
| Water | 53% |
| Glycerin | 10% |
| Xylitol | 4% |
| Propylene Glycol | 3% |
| Sodium Benzoate | 1% |
| Wintergreen Oil | 2% |
| Griffonia Simplicofolia | 1% |
| Garcina Cambogia | 4% |
| Kola Nut | 4% |
| Guarana | 4% |
| Yerba Mate | 4% |
| Myrrh Gum | 1% |
| Citrus Aurantium | 4% |
| Gymnema Sylvestre | 4% |
| Green Tea | 1%. |

SECOND EMBODIMENT OF
APPETITE SUPPRESSANT MOUTH SPRAY

| INGREDIENTS | PERCENT % |
|---|---|
| Water or other liquid carrier | up to 71% |
| Sweetener | 4% |
| Preservative | 3% |
| Wintergreen Oil | 2% |
| Griffonia Simplicofolia | 1% |
| Garcina Cambogia | 4% |
| Kola Nut | 4% |
| Guarana | 4% |
| Yerba Mate | 4% |
| Myrrh Gum | 1% |
| Citrus Aurantium | 4% |
| Gymnema Sylvestre | 4% |
| Green Tea | 1%. |

The mouth spray compositions illustrated above have intense flavor and suppress the appetite of the consumer after spraying their teeth and oral mucosa therewith. This provides a harmless topical manner of controlling weight without ingesting drugs, troches, lozenges, gums, or the like.

Variations in the above formulations may be made. For example, other food grade anionic or nonionic surfactants may be substituted. Similarly, high impact or intense flavoring compositions may be used in lieu of the oil of wintergreen, such as chocolate, mint, strawberry, grape or the like.

From the above description it can be seen that the appetite suppressant mouth spray of the present invention is able to overcome the shortcomings of prior art products by providing an appetite suppressant mouth spray by the incorporation of an appetite suppressant agent comprising natural herbs. The appetite suppressant mouth spray is preferably in the form of an oral hygenic liquid for use preferably prior to meals, to promote weight loss as a new secondary benefit to using a mouth spray. The appetite suppressant mouth spray promotes weight loss via a mouth spray formulation containing safe natural herbs. Furthermore, the appetite suppressant mouth spray of the present invention is simple and easy to use and economical in cost to manufacture.

A latitude of modification, change, and substitution is intended in the foregoing disclosure, and in some instances, some features of the invention will be employed without a corresponding use of other features. Accordingly, it is appropriate that the appended claims be construed broadly and in a manner consistent with the spirit and scope of the invention herein.

What is claimed is:

1. A mouth spray composition for suppressing appetite and promoting weight loss while simultaneously promoting oral intraoral health, comprising:
a) mouth spray base ingredients including Water; Glycerin; Xylitol; Propylene Glycol; Sodium Benzoate and Wintergreen Oil; and
b) a plurality of appetite suppressing and depressing herbs selected from the group consisting of Griffonia Simplicofolia; Garcina Cambogia; Kola Nut; Guarana; Yerba Mate; Myrrh Gum; Citrus Aurantium; Gymnema Sylvestre and Green Tea.

2. The composition of claim 1 wherein said mouth spray base ingredients include Water 53% to 54%; Glycerin 10%; Xylitol 4%; Propylene Glycol 3%; Sodium Benzoate 0.25% to 1% and Wintergreen Oil 2%.

3. The composition of claim 1 wherein said herbs are selected from the group consisting of Griffonia Simplicofolia 1%; Garcina Cambogia 4%; Kola Nut 4%; Guarana 4%; Yerba Mate 4%; Myrrh Gum 1%; Citrus Aurantium 4%; Gymnema Sylvestre 4% and Green Tea 1%.

4. The composition of claim 1 further including a high impact flavor of oil of wintergreen, chocolate, cherry, strawberry or grape.

5. A mouth spray composition for suppressing appetite and promoting weight loss while simultaneously promoting oral intraoral health, comprising:
a) mouth spray base ingredients including a mouth spray carrier, a sweetener, and a preservative; and
b) a plurality of appetite suppressing and depressing herbs selected from the group consisting of Griffonia Simplicofolia; Garcina Cambogia; Kola Nut; Guarana; Yerba Mata; Myrrh Gum; Citrus Aurantium; Gymnema Sylvestre and Green Tea.

6. The composition of claim 5 wherein said mouth spray base ingredients include water 53% to 54%; a sweetener of up to 4%, and a preservative of up to 1%.

7. The composition of claim 5 wherein said herbs are selected from the group consisting of Griffonia Simplicofolia 1%; Garcina Cambogia 4%; Kola Nut 4%; Guarana 4%; Yerba Mate 4%; Myrrh Gum 1%; Citrus Aurantium 4%; Gymnema Sylvestre 4% and Green Tea 1%.

8. The composition of claim 5 further including a high impact-flavor of oil of wintergreen, chocolate, cherry, strawberry or grape.
